(19) United States
(12) Patent Application Publication
(10) Pub. No.: US 2013/0325073 A1
(43) Pub. Date: Dec. 5, 2013

(54) Bone Anchors, Kits, and Methods for Securing Portions of Bone

(71) Applicants: George Sikora, Bridgewater, MA (US); Nikhil Jawrani, Framingham, MA (US)

(72) Inventors: George Sikora, Bridgewater, MA (US); Nikhil Jawrani, Framingham, MA (US)

(21) Appl. No.: 13/793,759

(22) Filed: Mar. 11, 2013

Related U.S. Application Data
(60) Provisional application No. 61/755,180, filed on Jan. 22, 2013.
(60) Provisional application No. 61/637,064, filed on Apr. 23, 2012.

(51) Int. Cl. A61B 17/80

(52) U.S. Cl. CPC A61B 17/80
USPC 606/286

(57) ABSTRACT

Embodiments of apparatuses, kits, and methods for securing portions of bone (e.g., for an osteotomy).

### CROSS-REFERENCE TO RELATED APPLICATIONS

This application claims priority to U.S. Provisional Patent Application Nos. 61/755,180, filed Jan. 22, 2013, and 61/637,064, filed Apr. 23, 2012, which are incorporated by reference in their entireties.

### BACKGROUND

1. Field of the Invention

The present invention relates generally to orthopedic treatments, and more particularly, but not by way of limitation, to devices, kits, and methods for securing portions of bone (e.g., for an osteotomy).

2. Description of Related Art

An example of an orthopedic fastener that can be used for an osteotomy is disclosed in U.S. Patent Application No. 10/165,332, filed Jun. 7, 2002 and published as Pub. No. US 2003/0228556. Another example of an orthopedic fastener is disclosed in U.S. Pat. No. 7,316,532.

### SUMMARY

This disclosure includes embodiments of bone-anchoring apparatuses, fastening apparatuses, kits, and methods.

Some embodiments of the present bone-anchoring apparatuses comprise: a bridge member having a first end, a second end, a first opening extending through the bridge member, and a second opening extending through the bridge member at a point between the first opening and the second end; and a (e.g., flexible) tension member coupled to the bridge member; where the apparatus is configured such that if a fastener extends through the first opening to secure the bridge member relative to a bone, the (e.g., flexible) tension member can be tensioned to position the second opening relative to the bone. In some embodiments, the member comprises a suture extending through the second opening. In some embodiments, the tension member comprises a wire extending through the second opening. In some embodiments, the first end of the bridge member is parallel to the second end of the bridge member. In some embodiments, the first end of the bridge member is not parallel to the second end of the bridge member. In some embodiments, the first end of the bridge member is disposed at angle of between 140 and 180 degrees relative the second end of the bridge member. In some embodiments,

In some embodiments of the present apparatuses, the bridge member comprises a proximal surface configured to face a bone and a distal surface configured to face away from a bone, and the first opening and the second opening each have a first transverse dimension and a second transverse dimension that is larger than the first transverse dimension and that is disposed between the first transverse dimension and the distal surface. In some embodiments, a central longitudinal axis of the first opening is disposed at a non-perpendicular angle relative to the first end of the bridge member, and a central longitudinal axis of the second opening is disposed at a non-perpendicular angle relative to the second end of the bridge member. In some embodiments, the non-perpendicular angle is between 90 degrees and 110 degrees. In some embodiments, the non-perpendicular angle is between 90 and 100 degrees. In some embodiments, the first opening and the second opening are each tapered. In some embodiments, the bridge member and the tension member are each sterile. In some embodiments, the bridge member comprises a proximal surface configured to face a bone and a distal surface configured to face away from a bone, and the bridge member includes one or more projections extending from the proximal surface in a direction away from the distal surface.

Some embodiments of the present fastening apparatuses comprise: a body comprising a handle portion, a first fastener portion extending from the handle portion, and a second fastener portion extending from the handle portion; where the first fastener portion and the second fastener portion each: includes one or more barbs or threads, and is configured to be inserted into a target material and to separate from the handle portion in response to movement of the handle portion relative to the fastener portion to break a material connecting the fastener portion to the handle portion. In some embodiments, the first fastener portion and the second fastener portion each is unitary with the handle portion. In some embodiments, the first fastener portion and second fastener portions each comprises an enlarged proximal end, a distal end, and a length extending from the proximal end and the distal end. In some embodiments, the body comprises a first connection portion between the handle portion and the first fastener portion, and a second connection portion between the handle portion and the first fastener portion, and where the first connection portion and the second connection portion each is configured to break in response to movement of the handle portion relative to the corresponding fastener portion. In some embodiments, the first connection portion and the second connection portion each has a diameter that is smaller than an adjacent diameter of the handle portion and an adjacent diameter of the corresponding fastener portion. In some embodiments, the first fastener portion and the second fastener portion each includes one or more threads. In some embodiments, the first fastener portion and second fastener portion each comprises a distal end configured to be rotated in contact with a bone to penetrate the bone. In some embodiments, the distal end of each of the first fastener portion and the second fastener portion comprises one or more cutting edges. In some embodiments, the first fastener portion and the second fastener portion each comprises a self-tapping screw. In some embodiments, a single straight axis extends through the body, the first fastener portion, and the second fastener portion.

Some embodiments of the present kits comprise: an embodiment of the present bone-anchoring apparatuses; and an embodiment of the present fastener apparatuses.

Some embodiments of the present methods comprise: tensioning the (e.g., flexible) tension member of an embodiment of the present bone-anchoring apparatuses as a first fastener extending through the first opening of the bridge member is inserted at least partially into a first portion of bone; and coupling the bridge member to a second portion of bone with a second fastener extending through the second opening of the bridge member. In some embodiments, coupling the bridge member to the second portion of bone comprises inserting the second fastener portion of an embodiment of the present fastener apparatuses through the second opening of the bridge member and into the second portion of bone. In some embodiments, the tension is maintained in the (e.g., flexible) tension member as the apparatus is coupled to the second portion of bone. In some embodiments, the first portion of bone and the second portion of bone are parts of the same bone. In some embodiments, an osteotomy separates the first portion of bone and the second portion of bone. Some embodiments further comprise: forming an osteotomy in a bone. Some embodiments further comprise: forming a hole in the second portion of bone prior to coupling the bridge member to the second portion of bone. Some embodiments further comprise: forming a hole in the first portion of bone prior to inserting the first fastener.

Any embodiment of any of the present apparatuses and methods can consist of or consist essentially of—rather than comprise/include/contain/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

Details associated with the embodiments described above and others are presented below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. The figures are drawn to scale (unless otherwise noted), meaning the sizes of the depicted elements are accurate relative to each other for at least the embodiments depicted in the figures.

FIG. 1 depicts a perspective view of an embodiment of the present bone-anchoring apparatuses comprising a bridge member and shown in combination with two fasteners.

FIGS. 2A-2E depict various views of the bridge member of FIG. 1.

FIGS. 3A-3D depict various views of a fastening apparatus for use with the bridge member of FIGS. 2A-2E.

FIGS. 4A-4G depict various views of stages of the present methods of use of the fastening apparatus of FIGS. 3A-3D with the bone-anchoring apparatus of FIG. 1.

FIG. 5A-5F depict various views of the method of FIGS. 4A-4G with an osteotomy in a bone.

FIGS. 6A-6C depict various views of a second embodiment of a bridge member for use with the present bone-anchoring apparatuses.

FIGS. 7A-7C depict various views of a third embodiment of a bridge member for use with the present bone-anchoring apparatuses.

### DESCRIPTION OF ILLUSTRATIVE EMBODIMENTS

The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art. In any embodiment of the present apparatuses, kits, and methods, the term “substantially” may be substituted with “within [a percentage] of” what is specified, where the percentage includes 0.1, 1, 5, and/or 10 percent.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, an apparatus or kit that “comprises,” “has,” “includes” or “contains” one or more elements possesses those one or more elements, but is not limited to possessing only those elements. Likewise, a method that “comprises,” “has,” “includes” or “contains” one or more steps possesses those one or more steps, but is not limited to possessing only those one or more steps.

Further, an apparatus, device, or structure that is configured in a certain way is configured in at least that way, but it can also be configured in other ways than those specifically described.

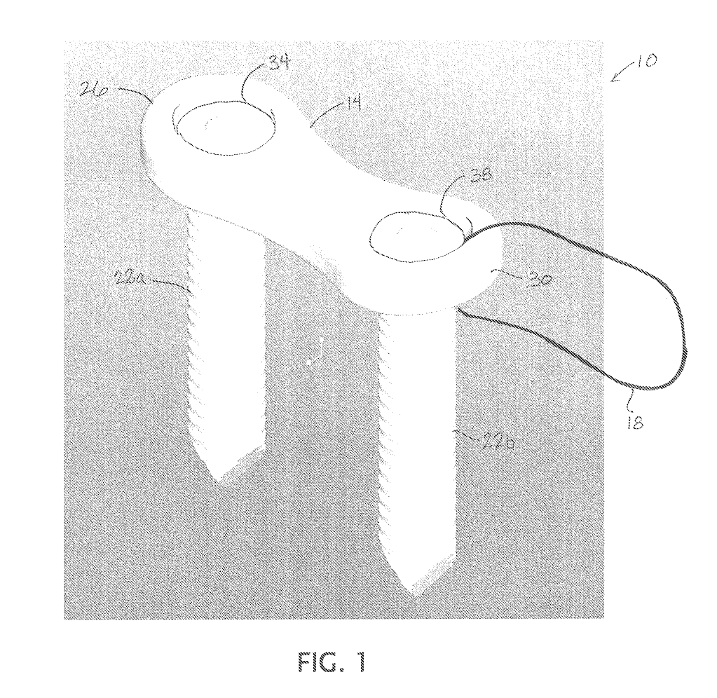

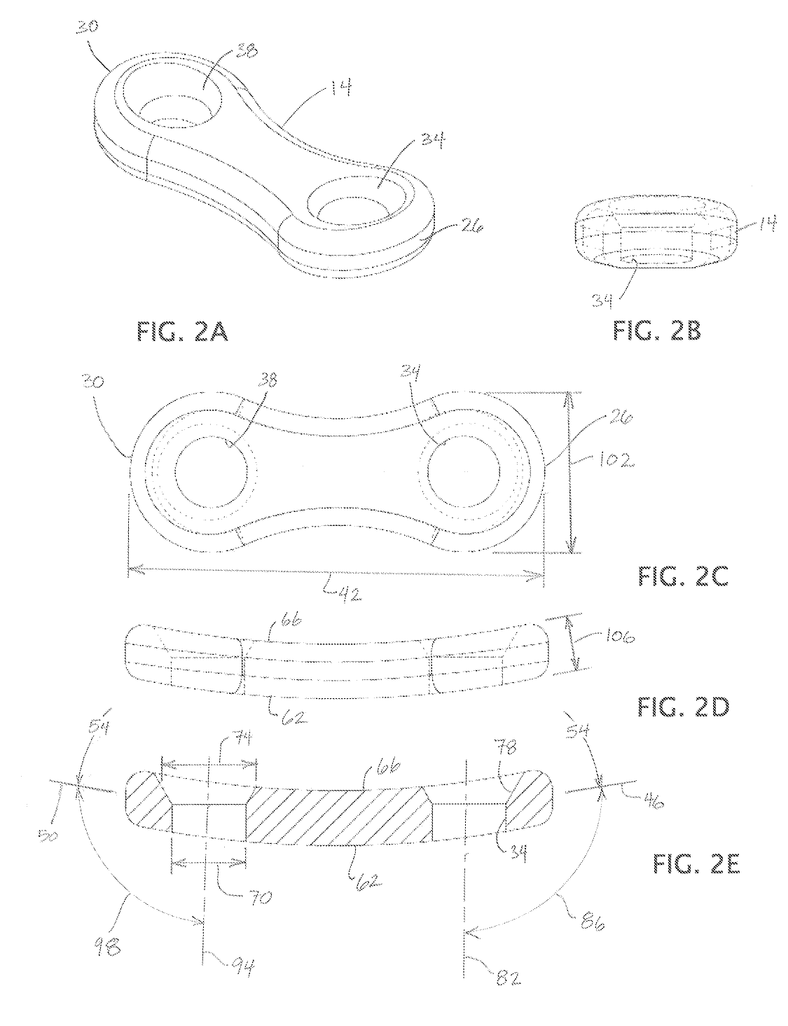

Referring now to the drawings, and more particularly to FIGS. 1 and 2A-2E, shown therein and designed by the reference numeral 10 is one embodiment of the present bone-anchoring apparatuses 10 comprising a bridge member 14 (which may also be characterized as a plate) and a (e.g., flexible) tension member 18 coupled to bridge member 14. FIG. 1 depicts apparatus 10 in combination with two fasteners 22a and 22b that can be used with bridge member 14 to secure two portions of bone (e.g., on opposing sides of an osteotomy). In the embodiment shown, bridge member 14 has a first end 26, a second end 30, a first opening 34 extending through the bridge member, and a second opening 38 extending through the bridge member at a point between first opening 34 and second end 30, as shown. In this embodiment, tension member 18 is coupled to bridge member 14 and apparatus 10 is configured such that a fastener (e.g., 22a) extends through first opening 34 to secure bridge member 14 relative to a bone, tension member 18 can be tensioned (e.g., pulled on by a user to generate traction) to maintain the position of tension member as the first fastener (22a, in this example) is inserted (e.g., driven) into bone (e.g., to stop the tension member from pivoting or rotating about the axis of the first fastener as the first fastener is driven into bone), position second opening 38 relative to the bone (e.g., to permit insertion of a second fastener (e.g., 22b) through second opening 38 and into the bone to secure bridge member 14), and/or to help compress the osteotomy. The tensioning or traction may be achieved by causing (e.g., traction) force to be applied to the bridge member through the tension member that is substantially parallel to the longitudinal axis (not shown) of the bridge member. In the embodiment shown, tension member 18 comprises a suture (e.g., a loop of suture, as shown) extending through second opening 38. In other embodiments, tension member can comprise a flexible wire (e.g., a loop of wire) extending through second opening 38. One type of suture suitable for use as tension member 18 is TiCron™ suture, available from Covidien, Mansfield, Mass., USA or Dublin, Ireland. One type of wire suitable for use as tension member 18 is a Nickel-Titanium allow wire loop (e.g., Nitinol). The tension member may have any suitable length; and when a loop is used, the loop of the tension member that is threaded through the bridge member may be positioned loosely (as shown) around the outer portion of the bridge member or, in other embodiments, may be positioned tightly around the outer portion of the bridge member. In other embodiments, tension member 18 is rigid (e.g., a rod or bar) or comprises one or more flexible element in combination with one or more rigid elements (e.g., a suture or wire in combination with a rod or bar).

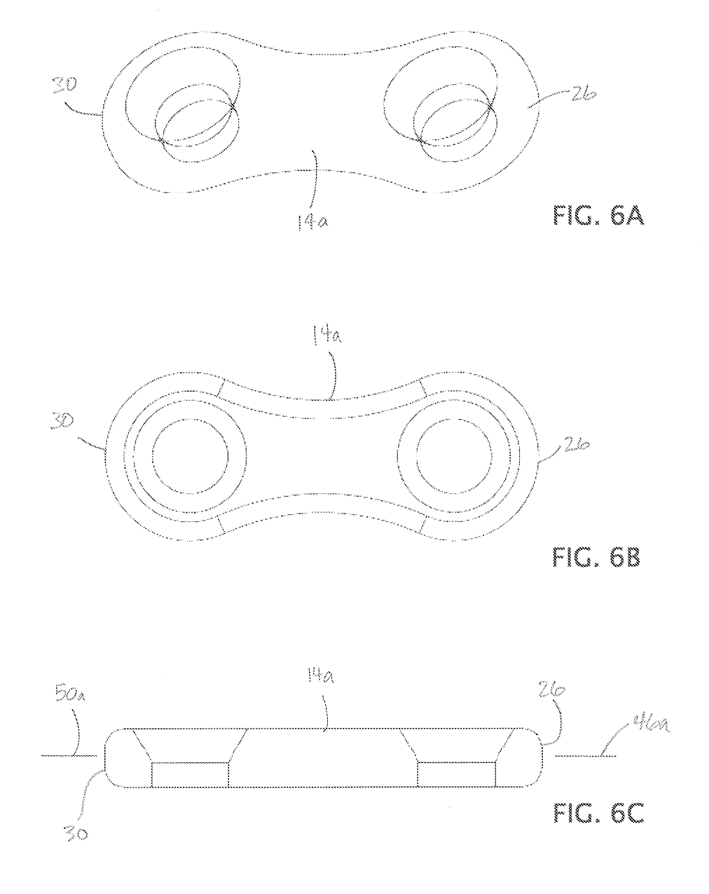

In the embodiment shown, first end 26 of the bridge member is not parallel to second end 30 of the bridge member. For example, in the embodiment shown, the bridge member has a length 42 extending between first end 26 and second end 30, and the bridge member is curved along its length. As used in this disclosure, “first end 26 is not parallel to second end 30” means that a line 46 projected either tangent to (for the curved embodiment shown) or parallel to (for a non-curved embodiment, such as is described below and depicted in FIGS. 6A-6C) a small portion of the length of the bridge member adjacent first end 26 is not parallel to a line 50 projected either tangent to (for the curved embodiment shown) or parallel to (for a non-curved embodiment, such as is described below and depicted in FIGS. 6A-6C) a small portion of the length of the bridge member adjacent second end 30. In some embodiments, the angle 54 between line 46 and line 50 is between 140 and 180 degrees (e.g., between 160 and 180 degrees).

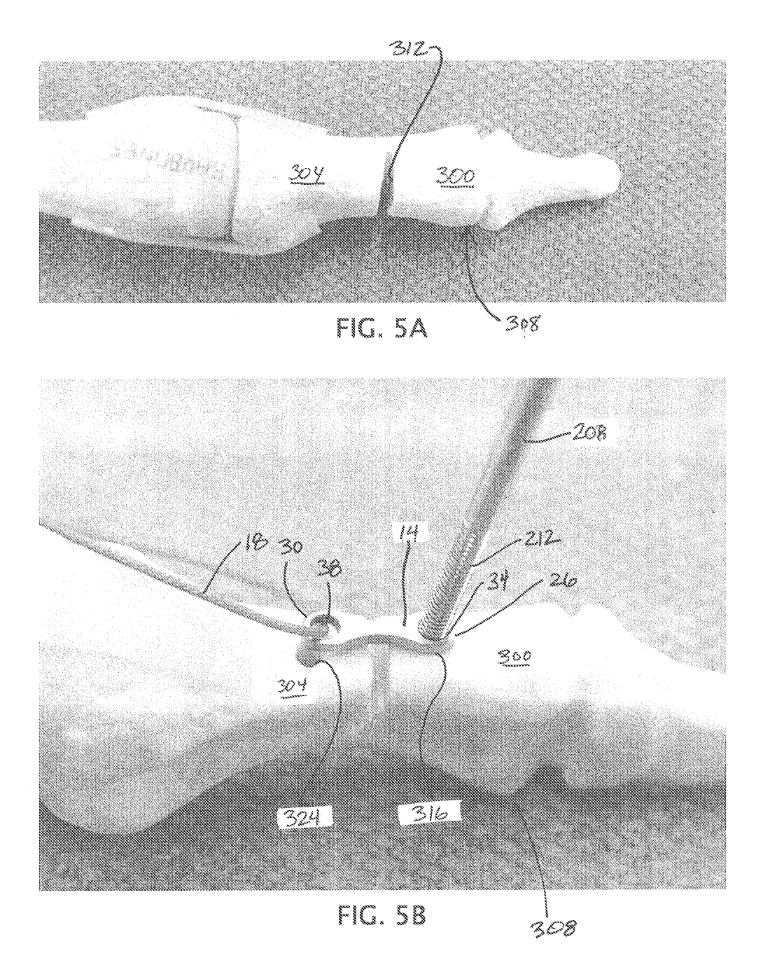

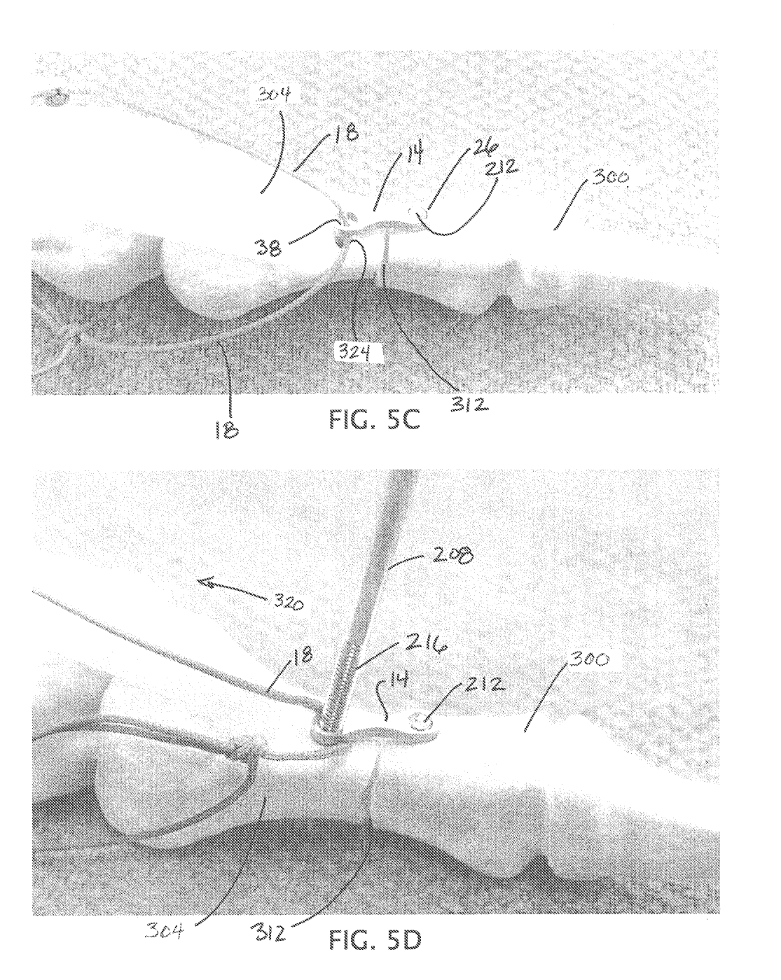

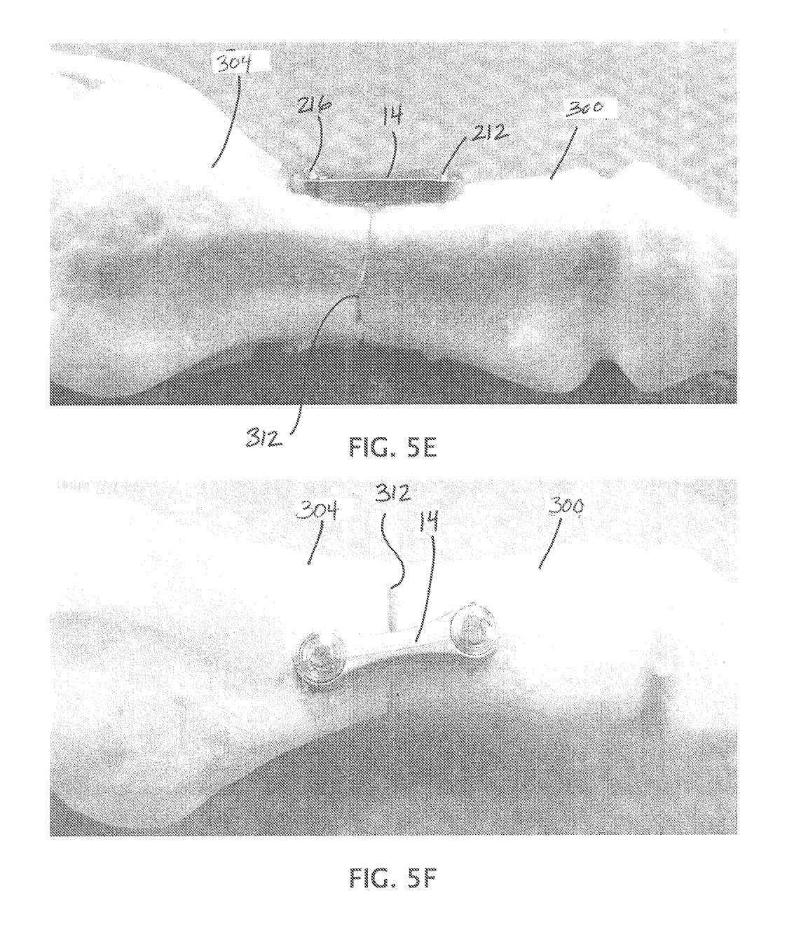

In some embodiments, bridge member 14 comprises a proximal surface 62 configured to face a bone (e.g., when the bridge member is coupled to a bone, as described below and depicted in FIGS. 5A-5F) and a distal surface 66 configured to face away from a bone, and first opening 34 and second opening 38 each have a first transverse dimension 70 and a second transverse dimension 74 that is larger than the first transverse dimension, where second transverse dimension 74 is disposed between first transverse dimension 70 and distal surface 66. For example, in the embodiment shown, openings 34 and 38 are tapered (include a tapered portion 78 adjacent distal surface 66, as shown). In this embodiment, larger dimension 74 can at least partially receive an enlarged head of a fastener (e.g., beneath distal surface 66) and smaller dimension 70 can prevent the enlarged head of the fastener from passing through the respective opening. In other embodiments, tapered portion 78 can comprise, alternatively or additionally, a shelf and/or the like.

The first and second openings, in some embodiments (including those shown herein) are not connected by a slot; in other embodiments, the first and second openings are connected by a slot.

In some embodiments in which bridge member 14 is curved or otherwise has non-parallel ends, such as the one shown, openings 34 and 38 may not be perpendicular to their respective adjacent ends. For example, in the embodiment shown, a straight, central longitudinal axis 82 of first opening 34 is disposed at a non-perpendicular angle 86 relative to first end 26 (relative to line 46), and a straight, central longitudinal axis 94 of second opening 38 is disposed at a non-perpendicular angle 98 relative to second end 30 (relative to line 50). In the embodiment shown, angle 86 is equal to angle 94, and both are between 90 degrees and 110 degrees (e.g., between 90 degrees and 100 degrees). In other embodiments, first opening 34 (axis 82) can be perpendicular to first end 26 (line 46) and/or second opening 38 (axis 94) can be perpendicular to second end 30 (line 50).

In the embodiment shown, bridge member 14 is configured such that upon tensioning to tension member 18 with sufficient force prior to inserting (e.g., driving in) the second fastener, tension in bridge member 14 will tend to draw together or apply an inward force on the two fasteners 22a and 22b (e.g., to provide a compressive force on an osteotomy between fasteners 22a and 22b).

In some embodiments, bridge member 14 is configured to fit within a rectangular volume of less than 500 millimeters cubed (mm³) (e.g., less than 400 mm³). For example, in some embodiments, member 14 has a length 42 of between 10 mm and 20 mm (e.g., greater than, or between any two of: 10, 12, 14, 16, 18, or 20 mm), a width 102 of between 3 mm and 10 mm (e.g., greater than, or between any two of: 3, 4, 5, 6, 7, 8, 9, or 10 mm), and/or a thickness 106 of between 1 mm and 2 mm (e.g., greater than, or between any two of: 1, 1.2, 1.4, 1.6, 1.8, or 2 mm).

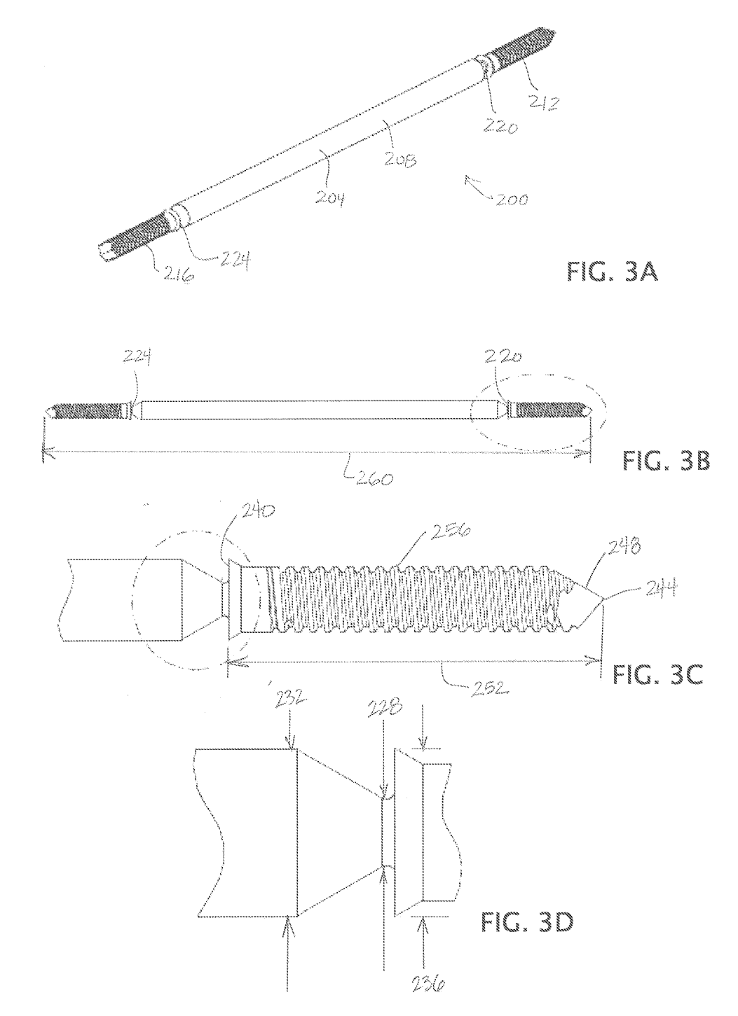

FIGS. 3A-3D depict various views of a fastening apparatus 200 for use with bridge member 14 of FIGS. 2A-2E. In the embodiment shown, apparatus 200 comprises: a body 204 comprising a handle portion 208, a first fastener portion 212 (which is an example of fastener 22a) extending from the handle portion, and a second fastener portion 216 (which is an example of fastener 22b) extending from the handle portion. In this embodiment, a single straight (e.g., central longitudinal) axis extends through the body, the first fastener portion, and the second fastener portion. In other words, in this embodiment, the body and the first and second fastener portions are aligned.

In this embodiment, each of fastener portions 212 and 216 is configured to be inserted into a target material (e.g., bone) and to separate from handle portion 208 in response to movement of the handle portion relative to the fastener portion to break a material connecting the fastener portion to the handle portion. For example, in the embodiment shown, body 204 comprises a first connection portion 220 between handle portion 208 and first fastener portion 212, and a second connection portion 224 between handle portion 208 and second fastener portion 216. In this embodiment, first connection portion 220 and second connection portion 224 each is configured to break in response to movement of handle portion 208 relative to the corresponding fastener portion 212 or 216. For example, in the embodiment shown, connection portions 220 and 224 each has a diameter 228 that is smaller than an adjacent diameter 232 of the handle portion and an adjacent diameter 236 of the corresponding fastening portion 212 or 216. In the embodiment shown, fastener portions 212 and 216 (and connection portions 220 and 224) are unitary with handle portion 208 (comprise a single piece of material). In other embodiments, fastener portions 212 and 216 can be coupled to handle portion 208 by any suitable structure or connection (e.g., corresponding or interlocking shapes, adhesive, and/or welds) that permits apparatus 200 to function as described in this disclosure (e.g., such that handle portion 208 and fastener portions 212 and 216 are functionally a single piece that can be together manipulated with a single hand of a user unless and until either or both of fastener portions 212 and 216 are broken off or otherwise separated from handle portion 208).

Fastener portions 212 and 216 can, for example, comprise one or more barbs or threads. For example, in the embodiment shown, fastener portions 212 and 216 are self-tapping screws that each includes an enlarged head or proximal end 240, a distal end 244 with one or more cutting edges 248, and a length 252 between head 240 and distal end 244. In this embodiment, distal ends 244 of fastener portions 212 and 216 are each configured to be rotated in contact with a bone to penetrate the bone (e.g., such that cutting edge(s) 248 will cut into the bone to facilitate the engagement of thread(s) 256 with the bone to secure the respective fastening portion with the bone). In this embodiment, pilot holes need not be separately drilled into the bone before inserting fastener portions 212 and/or 216. In other embodiments, fastener portions 212 and/or 216 can be configured to require a pilot hole (e.g., fastener portions 212 and 216 can be smooth pins and/or can have one or more barbs such that they are configured to be axially inserted into a hole rather than being rotated or screwed into the bone; or can include threads without cutting edges, such that a pilot hole is needed to screw or rotate the fastening portions into the bone). In some embodiments, length 252 of each fastener portion 212 and 216 is between 6 mm and 40 mm (e.g., greater than, or between any two of: 6, 10, 15, 20, 25, 30, 35, or 40 mm). In some embodiments, an overall length 260 of apparatus 200 is between 50 mm and 200 mm (e.g., greater than, or between any two of: 50, 75, 100, 125, 150, 175, or 200 mm), and handle portion 208 and/or enlarged head 240 have a transverse dimension (e.g., diameter) 232 and/or 236 of between 1 mm and 3 mm (e.g., greater than, or between any two of: 1, 1.25, 1.5, 1.75, 2, 2.25, 2.5, 2.75, or 3 mm).

Bridge member 14 and/or fastening apparatus 200 can comprise any biocompatible material(s) that permits apparatus 10 and apparatus 200 to function and/or be used in the manner described in this disclosure. For example, in some embodiments in which first end 26 is not parallel to second end 30 and/or some flexibility and/or resilience is desired for bridge member 14, bridge member 14 can comprise a biocompatible polymer (e.g., polyether ether ketone (PEEK)). In other embodiments in which rigidity is desired for bridge member 14, bridge member 14 can comprise a biocompatible metal (e.g., titanium, surgical stainless steel, and/or the like). Similarly, body 204 of fastening apparatus 200 can comprise a biocompatible polymer in embodiments in which pilot holes 316 and 324 are formed in bone or other target material prior to insertion of fastener portions 212 and 216, or body 204 can comprise a biocompatible metal in embodiments (such as the one the one shown) in which fastener portions 212 and 216 are configured as self-tapping screws.

Some embodiments of the present kits comprise an embodiment of the present bone-anchoring apparatuses (e.g., 10) and/or an embodiment of the present fastener apparatuses (e.g., 200). Some embodiments can comprise a tray or other container in a package (e.g., a sealed pouch or the like), in which the bone-anchoring apparatus and/or the fastening apparatus can be disposed for shipping and/or storage. In some embodiments, the bone-anchoring apparatus and/or the fastening apparatus are sterile. Both the tray and the package may be rectangular in shape. For example, some embodiments of the present kits may include a package (e.g., a box or a flexible package) that comprises sterilized versions of the bone-anchoring apparatus and/or the fastening apparatus. Embodiments of the present kits may also include, in more specific embodiments, instructions for use, which instructions may be inside the package (e.g., as an insert) or outside the package (such as a sticker on the package).

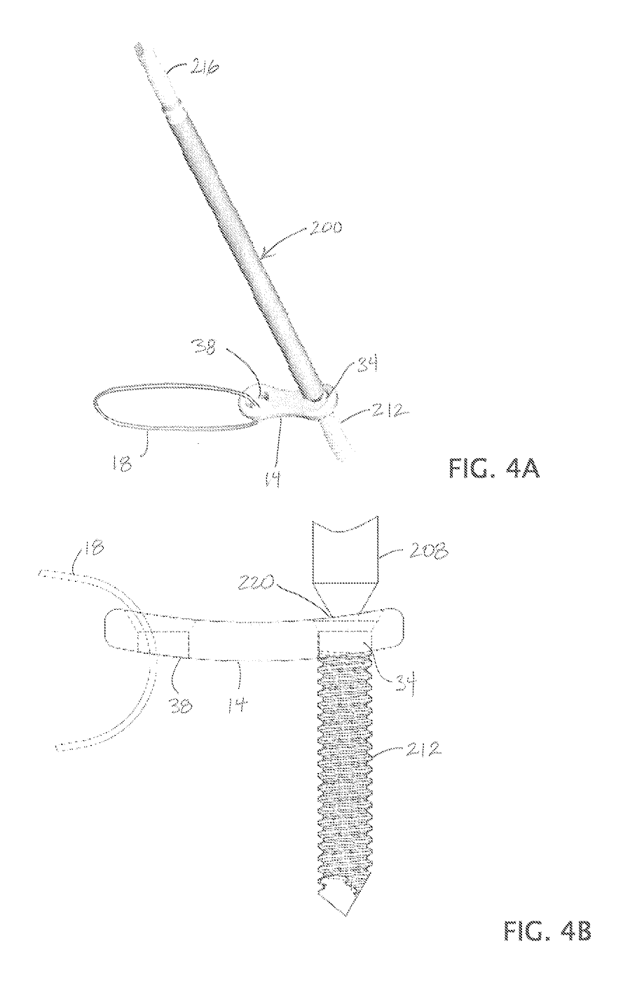

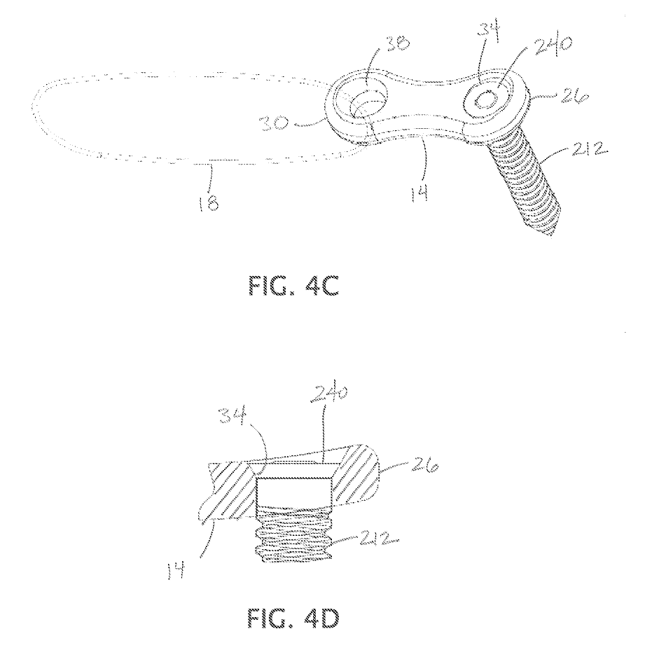

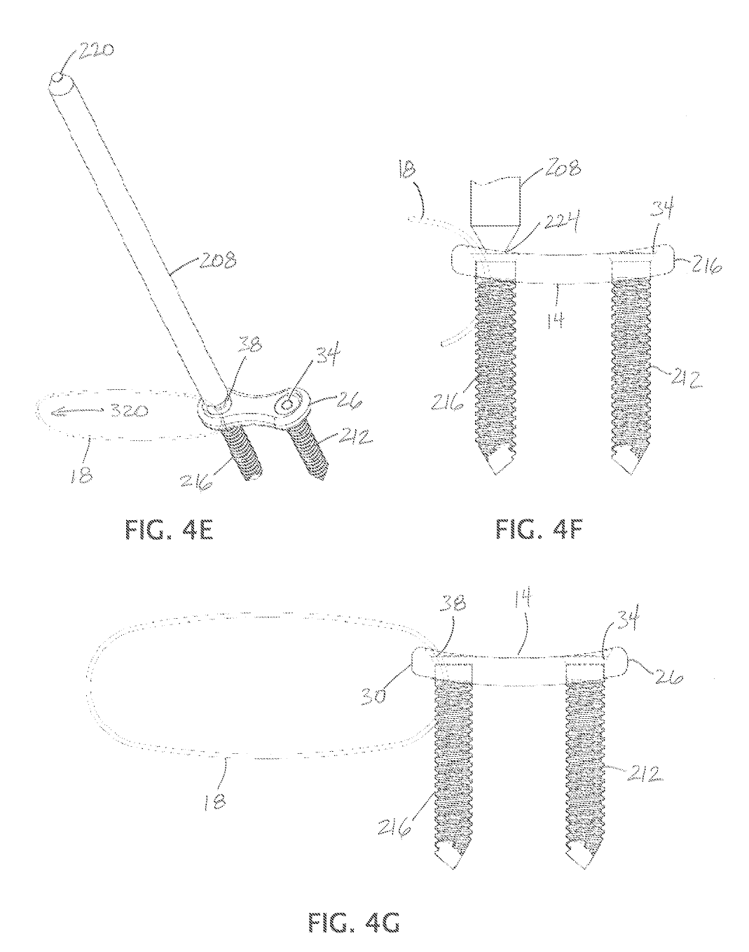

FIGS. 4A-4G depict various views of stages of the present methods of use of the fastening apparatus of FIGS. 3A-3D with the bone-anchoring apparatus of FIG. 1; and FIG. 5A-5F depict various views of the method of FIGS. 4A-4G with an osteotomy in a bone. In some embodiments of the present methods, first end 26 of bridge member 14 is coupled to a first portion 300 of bone, and second end 30 of bridge member 14 is coupled to a second portion 304 of bone. For example, first portion 300 and second portion 304 can be parts of a single bone 308 and/or may be separated by an osteotomy 312, as shown in FIG. 5A. Some embodiments of the present methods comprise forming an osteotomy 312 (e.g., a V-shaped osteotomy 312, as shown in FIG. 5A) in a bone (e.g., a phalange proximales or toe bone, as shown (which may also be referred to as the proximal phalanx)).

In some embodiments, the method further comprises coupling bridge member 14 to first portion 300 with a fastener extending through first opening 34 of the bridge member. For example, in the embodiment shown, first fastener portion 212 of apparatus 200 is inserted through first opening 34 of bridge member 14 (as shown in FIGS. 4A and 4B) and into first portion 300 (as shown in FIG. 5B). In some embodiments, tension member 18 can be held or tensioned during coupling of bridge member 14 to first portion 300 for one or more of the reasons set forth above. Some embodiments comprise forming a hole 316 in first portion 300 (to receive first fastener portion 212) prior to coupling bridge member 14 to first portion 300. However, in the embodiment of apparatus 200 that is shown, the step of forming hole 316 in first portion 300 is unnecessary because first fastener portion 212 is configured to be self-tapping, as described above, such that first fastener portion 212 can be rotated directly into undisturbed bone. Once first fastener portion 212 is fully seated into first portion 300 of bone 308, handle portion 208 can then be twisted and/or bent relative to first fastener portion 212 to break first connection portion 220 and thereby separate handle portion 208 from first fastener portion 212 (as shown in FIGS. 4C and 4D), and leave first fastener portion 212 securely seated in first portion 300 (as shown in FIG. 5C).

Some embodiments of the present methods comprise: tensioning tension member 18 (e.g., pulling member 18 in a direction 320 away from first opening 34 to close osteotomy 312 and/or compress first portion 300 and second portion 304 relative to each other) and/or coupling bridge member 14 to second portion 304 of bone with a fastener extending through second opening 38 of the bridge member. For example, in the embodiment shown, second fastener portion 216 of apparatus 200 is inserted through second opening 38 of bridge member 14 (as shown in FIGS. 4E and 4F) and into second portion 304 (as shown in FIG. 5D). In some embodiments, tension member 18 can be held or tensioned during coupling of bridge member 14 to second portion 304 for one or more of the reasons set forth above. Some embodiments comprise forming a hole 324 in second portion 304 (to receive second fastener portion 216) prior to coupling bridge member 14 to second portion 304. However, in the embodiment of apparatus 200 that is shown, the step of forming hole 324 in second portion 304 is unnecessary because second fastener portion 216 is configured to be self-tapping, as described above, such that second fastener portion 216 can be rotated directly into undisturbed bone. Once second fastener portion 216 is fully seated into second portion 304 of bone 308, handle portion 208 can then be twisted and/or bent relative to second fastener portion 216 to break second connection portion 224 and thereby separate handle portion 208 from second fastener portion 216 (as shown in FIG. 4G), and leave second fastener portion 216 securely seated in second portion 304 (as shown in FIGS. 5E and 5F). In some embodiments, tension member 18 will be cut as second fastener portion 216 is driven into second portion 304 (e.g., through interference between second fastener portion 216 and second portion 304 of the bone) or can otherwise be removed from bridge member 14 after coupling the bridge member to second portion 304.

FIGS. 6A-6C depict various views of a second embodiment 14a of a bridge member for use with the present bone-anchoring apparatuses. In the embodiment shown, bridge member 14a is substantially similar to bridge member 14, with the exception that in first end 26 (line 46a) of bridge member 14a is parallel to second end 30 (line 50a) of bridge member 14a.

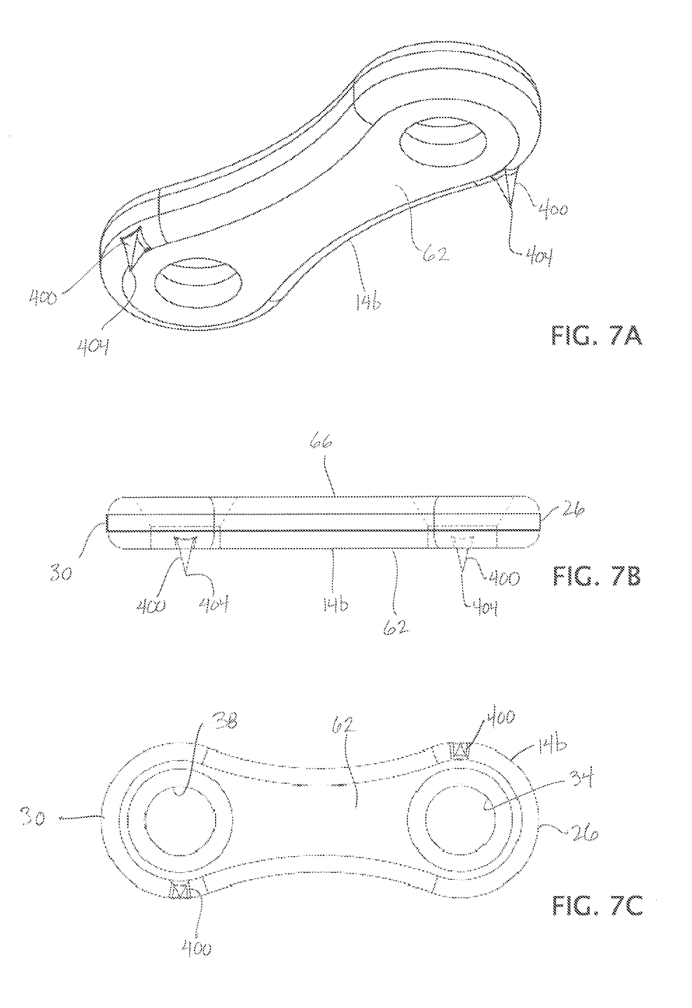

FIGS. 7A-7C depict various views of a third embodiment 14b of a bridge member for use with the present bone-anchoring apparatuses. In the embodiment shown, bridge member 14b is substantially similar to bridge member 14a, with the exception that bridge member 14b comprises one or more (e.g., two, as shown) protrusions 400 extending from proximal surface 62 in a direction away from distal surface 66. In the embodiment shown, protrusions 400 are pointed (e.g., each comprise sharpened point 404). In this embodiment, protrusions 400 are configured to contact bone to resist rotation of bridge member 14b relative to the bone (e.g., during insertion of fasteners such as, for example, 212 and/or 216). For example, in this embodiment, the protrusion that is closest to first end 26 is spaced apart from first opening 34 to contact first portion 300 of bone (or tissue adjacent to and/or overlying first portion 300), and the protrusion that is closest to second end 30 is spaced apart from second opening 38 to contact second portion 304 of bone (or tissue adjacent to and/or overlying second portion 304).

The above specification and examples provide a complete description of the structure and use of exemplary embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the present apparatuses are not intended to be limited to the particular forms disclosed. Rather, they include all modifications and alternatives falling within the scope of the claims, and embodiments other than the one shown may include some or all of the features of the depicted embodiment. For example, handle portion 208 of fastening apparatus 200 can have any suitable cross-sectional shape (e.g., triangular, square, rectangular, and/or the like) that permits the present apparatuses and methods to function as described in this disclosure. For example, components may be combined as a unitary structure, and/or connections may be substituted. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. A bone-anchoring apparatus comprising:
a bridge member having a first end, a second end, a first opening extending through the bridge member, and a second opening extending through the bridge member at a point between the first opening and the second end; and
a tension member coupled to the bridge member;
where the apparatus is configured such that if a fastener extends through the first opening to secure the bridge member relative to a bone, the tension member can be tensioned to position the second opening relative to the bone.

2. The apparatus of claim 1, where the tension member comprises a suture extending through the second opening.

3. The apparatus of claim 1, where the tension member comprises a tension wire extending through the second opening.

4. (canceled)

5. The apparatus of claim 1, where the first end of the bridge member is not parallel to the second end of the bridge member.

6. The apparatus of claim 5, where the first end of the bridge member is disposed at angle of between 140 and 180 degrees relative the second end of the bridge member.

7. The apparatus of claim 5, where the bridge member comprises a proximal surface configured to face a bone and a distal surface configured to face away from a bone, and the first opening and the second opening each have a first transverse dimension and a second transverse dimension that is larger than the first transverse dimension and that is disposed between the first transverse dimension and the distal surface.

8. The apparatus of claim 7, where a central longitudinal axis of the first opening is disposed at a non-perpendicular angle relative to the first end of the bridge member, and a central longitudinal axis of the second opening is disposed at a non-perpendicular angle relative to the second end of the bridge member.

9. The apparatus of claim 8, where the non-perpendicular angle is between 90 degrees and 110 degrees.

10-11. (canceled)

12. The apparatus of claim 1, where the bridge member and the tension member are each sterile.

13. The apparatus of claim 1, where the bridge member comprises a proximal surface configured to face a bone and a distal surface configured to face away from a bone, and the bridge member includes one or more projections extending from the proximal surface in a direction away from the distal surface.

14. A fastening apparatus comprising:
a body comprising a handle portion, a first fastener portion extending from the handle portion, and a second fastener portion extending from the handle portion;
where the first fastener portion and the second fastener portion each:
includes one or more barbs or threads, and
is configured to be inserted into a target material and to separate from the handle portion in response to movement of the handle portion relative to the fastener portion to break a material connecting the fastener portion to the handle portion.

15. The apparatus of claim 14, where the first fastener portion and the second fastener portion each is unitary with the handle portion.

16. The apparatus of claim 15, where the first fastener portion and second fastener portions each comprises an enlarged proximal end, a distal end, and a length extending from the proximal end and the distal end.

17. The apparatus of claim 14, where the body comprises a first connection portion between the handle portion and the first fastener portion, and a second connection portion between the handle portion and the first fastener portion, and where the first connection portion and the second connection portion each is configured to break in response to movement of the handle portion relative to the corresponding fastener portion.

18. (canceled)

19. The apparatus of claim 14, where the first fastener portion and the second fastener portion each includes one or more threads.

20. The apparatus of claim 19, where the first fastener portion and second fastener portion each comprises a distal end configured to be rotated in contact with a bone to penetrate the bone.

21-22. (canceled)

23. The apparatus of claim 14, where a single straight axis extends through the body, the first fastener portion, and the second fastener portion.

24. A kit comprising:
a bone-anchoring apparatus of claim 1; and
a fastener apparatus of claim 14.

25. A method comprising:
tensioning the tension member of a bone-anchoring apparatus of claim 1 while a first fastener extending through the first opening of the bridge member is inserted at least partially into a first portion of bone; and
coupling the bridge member to a second portion of bone with a second fastener extending through the second opening of the bridge member.

26. The method of claim 25, where coupling the bridge member to the second portion of bone comprises inserting the second fastener portion of a fastener apparatus of claim 14 through the second opening of the bridge member and into the second portion of bone.

27. The method of claim 25, where the tension is maintained in the tension member as the apparatus is coupled to the second portion of bone.

28. The method of claim 27, where the first portion of bone and the second portion of bone are parts of the same bone.

29. The method of claim 28, where an osteotomy separates the first portion of bone and the second portion of bone.

30-32. (canceled)
